(19) United States
(12) Patent Application Publication
(10) Pub. No.: US 2008/0153897 A1
(43) Pub. Date: Jun. 26, 2008

(54) CREATINE PYROGLUTAMIC ACID SALTS AND METHODS FOR THEIR PRODUCTION AND USE IN INDIVIDUALS

(75) Inventors: Michele Molino, Mississauga (CA); Shan Chaudhuri, Brampton (CA)

Correspondence Address: TORYS LLP, 79 WELLINGTON ST. WEST, SUITE 3000, TORONTO, ON, M5K 1N2, omitted

(73) Assignee: NEW CELL FORMULATIONS LTD., Mississauga (CA)

(21) Appl. No.: 11/962,929

(22) Filed: Dec. 21, 2007

Related U.S. Application Data
(63) Continuation-in-part of application No. 11/530,601, filed on Sep. 11, 2006, now Pat. No. 7,329,763.
Application No. 11/530,601 is a continuation-in-part of application No. 11/778,981, filed on Jul. 17, 2007.
Application No. 11/778,981 is a division of application No. 11/530,601, filed on Sep. 11, 2006, now Pat. No. 7,329,763.

(51) Int. Cl. A61K 31/4015; A61P 25/00

(52) U.S. Cl. 514/423

(57) ABSTRACT

A method for the use of creatine pyroglutamic acid salts is provided by the present invention. The salt of the present invention act substantially simultaneously to increase the neural health of a mammal.

### RELATED APPLICATIONS

This application is a continuation-in-part of U.S. patent application Ser. No. 11/530,601, entitled “Creatine pyroglutamic acid salts and method for their production and use in individuals,” filed on Sep. 11, 2006, and a continuation-in-part of U.S. patent application Ser. No. 11/778,981, which is a divisional application of U.S. patent application Ser. No. 11/530,601, the disclosures of which are hereby fully incorporated by reference herein.

### FIELD OF THE INVENTION

The present invention relates to a method of use for hydrosoluble stable organic salts of creatine and pyroglutamic acid. The present invention discloses a method for facilitating increased neural health in a mammal.

### BACKGROUND OF THE INVENTION

Creatine monohydrate is a commonly used supplement. Creatine monohydrate is soluble in water at a rate of 75 ml of water per gram of creatine. Ingestion of creatine monohydrate thereof requires large amounts of water to also be ingested. Additionally, in aqueous solutions, creatine converts to creatinine via an irreversible, pH-dependent, non-enzymatic reaction. Aqueous and alkaline solutions contain an equilibrium mixture of creatine and creatinine. In acidic solutions, on the other hand, the formation of creatinine is complete. Creatinine is devoid of the ergogenic beneficial effects of creatine and is typically excreted in the urine. It is therefore desirable to provide, for use in individuals, e.g. animals and humans, forms and derivatives of creatine with improved characteristics such as stability and solubility. Furthermore, it would be advantageous to do so in a manner that provides additional functionality compared to creatine monohydrate alone.

Hydrosoluble creatine monohydrate salts are obtainable and have been described elsewhere. For instance, U.S. Pat. No. 5,973,199, incorporated herein in its entirety by reference, purports to describe hydrosoluble organic salts of creatine as single combination of one mole of creatine monohydrate with one mole of the following organic acids: citrate, malate, fumarate, tartarate, and malate.

U.S. Pat. No. 5,925,278, incorporated herein in its entirety by reference, purports to describe a form of a creatine salt as a combination of one mole of creatine with one mole of citric acid.

U.S. Pat. No. 6,211,407, incorporated herein in its entirety by reference, purports to describe dicreatine and tricreatine citrate and methods of making the same. Salts are reported to be a combination of two and three moles of creatine monohydrate with one mole of citric acid, respectively. In addition, dicreatine and tricreatine citrate are claimed to be stable in acidic solution, in a guise to prevent or impede the formulation of creatine to creatinine.

U.S. Pat. No. 6,166,249, incorporated herein in its entirety by reference, purports to describe a creatine pyruvic acid salt where the ratio of creatine to pyruvate is 1:1 and the creatine pyruvate contains 1-10 molecules of water.

U.S. Pat. No. 5,973,199, incorporated herein in its entirety by reference, purports to describe a method of producing a creatine malate salt with a melting point of between 128 and 129° C. The patent also purports to describe a method of producing a creatine citrate salt with a melting point between 112 and 114° C.

U.S. Pat. No. 6,838,562, incorporated herein in its entirety by reference, purports to describe a process for the synthesis of mono, di, or tricreatine orotic acid, thioorotic acid, and dihydroorotic acid salts.

U.S. Pat. No. 6,861,554, incorporated herein in its entirety by reference, purports to describe a formula, a novel salt, creatine taurinate, and the compositions containing same (health foods, compositions or drugs).

### SUMMARY OF THE INVENTION

The present invention is directed towards the a method of use of a nutritional supplement, comprising at least a hydrosoluble stable organic salt of creatine and D,L-pyroglutamic acid, i.e. creatine pyroglutamate, characterized by high water solubility, i.e. from 2 to 25 times higher than that of creatine itself. The ingredients of the present nutritional supplement act to facilitate increased neural health in a mammal.

### DETAILED DESCRIPTION OF THE INVENTION

In the following description, for the purposes of explanation, numerous specific details are set forth in order to provide a thorough understanding of the present invention. It will be apparent, however, to one skilled in the art that the present invention may be practiced without these specific details.

The present invention is directed towards the administration of a creatine pyroglutamic acid salt to a mammal; specific benefits are conferred by both the creatine component and the pyroglutamate component. The preferred route of administration is oral. Disclosed in the description of the present invention is a use of creatine pyroglutamate in producing compositions for oral administration to provide benefits related to facilitating increased neural health in a mammal. Furthermore, the present invention is particularly well suited for use in tablets, capsules and solutions.

As used herein, ‘cognitive functions’ refers to any mental component of brain function. For example, cognitive functions include, but are not limited to, attention, concentration, memory and focus.

As used herein, ‘Creatine’ refers to the chemical N-methyl-N-guanyl Glycine, (CAS Registry No. 57-00-1), also known as, (alpha-methyl guanido) acetic acid, N-(aminoiminomethyl)-N-glycine, Methylglycocyamine, Methylguanidoacetic Acid, or N-Methyl-N-guanylglycine. Additionally, as used herein, ‘creatine’ also includes derivatives of creatine such as esters, and amides, and salts, as well as other derivatives, including derivatives having substantially similar pharmacoproperties to creatine upon metabolism to an active form.

### Creatine

Creatine is a naturally occurring amino acid derived from the amino acids glycine, arginine, and methionine. Although it is found in meat and fish, it is also synthesized by humans. Creatine is predominantly used as a fuel source in muscle. About 65% of Creatine is stored in muscle as Phosphocreatine (Creatine bound to a Phosphate molecule). Muscular contractions are fueled by the dephosphorylation of adenosine triphosphate (ATP) to produce adenosine diphosphate (ADP) and without a mechanism to replenish ATP stores, the supply of ATP would be totally consumed in 1-2 seconds. Phosphocreatine serves as a major source of Phosphate from which ADP is regenerated to ATP. Six seconds following the commencement of exercise, muscular concentrations of Phosphocreatine drop by almost 50% and Creatine supplementation has been shown to increase the concentration of Creatine in the muscle and further said supplementation enables an increase in the resynthesis of Phosphocreatine leading to a rapid replenishment of ATP within the first two minutes following the commencement of exercise. It may be through this mechanism that Creatine can improve strength and reduce fatigue.

Creatine also mediates remarkable neuroprotection in experimental models of amyotrophic lateral sclerosis, Huntington's disease, Parkinson's disease, and traumatic brain injury. Also, oral creatine administration to experimental animals has been shown to increase protection and inhibition of cell-death cascades in neural tissue during periods of stress, such as ischemia. The protective effect of creatine administration has been linked to improved energy balance. This increased energy balance, preservation of ATP levels, inhibits cytochrome c release from the mitochondria. Release of cytochrome c signals caspase-3 activation and cell death often results. Administration of creatine conserves energy balance in neural cells during periods of stress, thereby preserving neural tissue by inhibiting caspase-mediated cell death.

### Pyroglutamic Acid

Pyroglutamic acid (CAS 98-79-3) is a naturally occurring amino acid derived from L-glutamic acid and involved in glutathione metabolism. Pyroglutamic acid crosses the blood-brain barrier and is found in high levels in the brain where it is thought to act in improving cognitive function. Pyroglutamate is generally available as arginine pyroglutamate wherein, the primary claim made for this arginine salt of pyroglutamic acid relates to its use in helping overcome memory defects induced by alcohol abuse and in those with some forms of dementia. Pyroglutamic acid has been shown to improve specific aspects of cognitive function in rats. In humans pyroglutamic acid improves age-associated memory impairment.

N-terminal pyroglutamate formation from its glutaminyl precursor is an important posttranslational or cotranslational event in the processing of numerous bioactive neuropeptides, hormones, and cytokines. The N-terminal pyroglutamate is required by these regulatory peptides to develop the proper conformation for binding to their receptors, and to protect the peptides from degradation. This N-terminal cyclization reaction is catalyzed by the enzyme glutaminyl cyclase, which is responsible for posttranslational modification.

Interestingly, glutaminyl cyclase also catalyzes the N-terminal glutamate cyclization into pyroglutamate. This reaction is related to the formation of several plaque-forming peptides, such as amyloid-β (Aβ peptides, which are major peptides within the core of neuritic plaques. N-terminal pyroglutamate could enhance the hydrophobicity, proteolytic stability, and neurotoxicity of these peptides, thereby causing the accumulation of these Aβ peptides in senile plaques and quickening the progression of neurodegeration.

It is herein understood by the inventor that administration of pyroglutamate, in the form of a creatine salt, for example, will act as a feedback inhibitor to slow down the efficacy of glutaminyl cyclase in catalyzing the N-terminal glutamate cyclization into pyroglutamate, thereby preventing the acquisition of hydrophobic, proteolytic stability and neurotoxic characteristics by the Aβ peptides.

### Creatine Pyroglutamate

Creatine Pyroglutamate combines the muscle-enhancing and neuroprotective effects of creatine with the cognition-enhancing activity afforded by pyroglutamic acid. The novel organic compound can be used in sports nutrition as an ergogenic aid to increase strength, muscle volume and size, while affording improved capacity of concentration and mental focus during physical exertion. Also, this creatine salt can find employment in metabolic nutrition by defending against ischemic brain infarction and affording neuroprotection after cerebral ischemia.

Creatine pyroglutamic acid salts are used advantageously alone or with additional active ingredients, such as, trace elements, vitamins, mineral substances, or other amino acids as well as, optionally, excipients usually used for the preparation of the respective forms of administration. The forms of administration include, particularly, all varieties of tablets, both those that are swallowed without being chewed, and tablets to be chewed or dissolved in the mouth of an individual, as well as those that are dissolved in a liquid before being ingested by an individual. The tablet forms include uncoated tablets, one-layer or multilayer or encased forms or effervescent tablets. Further preferred forms of administration are capsules of hard and soft gelatin, the latter having particularly suitable to include a liquid core. Additionally, Creatine pyroglutamic acid salts can be used advantageously for the preparation of solutions and suspensions and as a powder, either effervescent or granulated.

It is understood by the inventors that creatine pyroglutamic acid salts are useful compounds, since they combine within a single molecule both the creatine and the pyroglutamate, thus resulting in the increase of the useful activities of these two compounds. Particularly, it is herein understood by the inventors that creatine pyroglutamic acid salts will have enhanced increased solubility.

Additionally, it is herein understood by the inventors that the creatine component of the salt will act to preserve cellular bioenergetics and inhibit caspase-mediated cell-death pathway activation. This preservation of ATP levels inhibits cytochrome c release from the mitochondria, thereby reducing caspase-3 activation and cell death thus preserving neural tissue.

Further to the aforementioned functions, it is also understood by the inventors that the pyroglutamate component of the salt will act as a feedback inhibitor to slow down the efficacy of glutaminyl cyclase in catalyzing the N-terminal glutamate cyclization into pyroglutamate, thereby preventing the acquisition of hydrophobic, proteolytic stability and neurotoxic characteristics by the Aβ peptides.

Furthermore, it is herein understood by the inventors that the components of the present invention will act in concert through at least the aforementioned distinct mechanisms to facilitate increased neural health in a mammal.

Although the following example illustrates the practice of the present invention in one of its embodiments, the example should not be construed as limiting the scope of the invention. Other embodiments will be apparent to one of skill in the art from consideration of the specifications and example.

### EXAMPLES

### Example 1

A nutritional supplement comprising the following ingredients per serving is prepared for consumption as four caplets to be administered in the morning:
About 1.500 g of a creatine pyroglutamic acid salt.

### Example 2

A nutritional supplement comprising the following ingredients per serving is prepared for consumption as a powder to be mixed with water and consumed in the morning:
About 1.500 g of a creatine pyroglutamic acid salt, and about 3.500 g of dextrose.

### Example 3

A nutritional supplement comprising the following ingredients per serving is prepared for consumption as three softgel capsules to be taken in the morning:
About 1.500 g of a creatine pyroglutamic acid salt, about 50 mg of ethyl pyruvate, and about 50 mg of geranylgeranylacetone.

What is claimed:

1. A method for facilitating increased neural health in a mammal comprising the step of administering to said mammal a composition comprising a creatine pyroglutamic acid salt.

2. The method of claim 1, wherein the increased neural health results in improved cognitive functions in the mammal.

3. The method of claim 1, wherein said administration prevents and fights the onset of stress and its deleterious effects on mental and physical performances.
